(19) United States
(12) Patent Application Publication
(10) Pub. No.: US 2006/0014695 A1
(43) Pub. Date: Jan. 19, 2006

(54) Hpma-polyamine conjugates and uses therefore

(76) Inventors: Hamidreza Ghandehari, Ellicott city, MD (US); Martin C. Woodle, Bethesda, MD (US); Puthupparampil V. Scaria, Montgomery Village, MD (US); Anjan Nan, Baltimore, MD (US)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/502,985

(22) PCT Filed: Jan. 31, 2003
(86) PCT No.: PCT/US03/02707

§ 371 Date: Aug. 16, 2005

Related U.S. Application Data
(60) Provisional application No. 60/352,883, filed on Feb. 1, 2002.

(51) Int. Cl. A61K 48/00; A61K 39/395

(52) U.S. Cl. 514/17; 514/44; 424/178.1

(57) ABSTRACT

The inventions provide compositions and methods for nucleic acid delivery comprising IIPMA conjugated to a polyamine. These compositions have the benefit of the steric hindrance of HPMA and the nucleic acid binding capability of a polyamine. Useful polyamines for this purpose include spermine, spermidine and their analogues, and DFMO. These polyamines have the ability not only to bind nucleic acids, but also have anti-cancer effects themselves. The compounds provided can also include ligand binding domains, such as vascular endothelial growth factors, somatostatin and somatostatin analogs, transferring, melanotropin, ApoE and ApoE peptides, von Willebrand's factor and von Willebrand's factor peptides, adenoviral fiber protein and adenoviral fiber protein peptides, PD 1 and PD 1 peptides, EGF and EGF peptides, RGD peptides, CCK peptides, antibody and antibody fragments, folate, pyridoxyl and sialyl-LewisX and chemical analogs. Methods for using these compositions to achieve a therapeutic effect, including for vaccination, are also provided.

### BACKGROUND OF THE INVENTION

1. Field of the Invention

The invention provides compositions for nucleic acid delivery comprising N-2-hydroxypropyl methacrylamide (HPMA) conjugated to a polyamine, and to methods for delivering one or more nucleic acids to a cell utilizing said compositions.

2. Background of the Invention

Gene delivery in therapeutic applications has long been plagued by a variety of complications. One problem is the rapid clearance from blood, leading to inactivation and excretion. Use of vectors such as viral vectors, polymers, nanoparticles, and lisosomes has been partly successful in addressing this problem, but these vectors raise additional difficulties, including immunogenicity and toxicity. This is especially problematic when using vectors for vaccines, which require repeated, or booster, doses of a particular antigen. Viral vectors, for example, are produced via a natural packaging cell production. Such “natural packaging” produces particles virtually identical to those of the virus from which the vector is derived. The produced capsid or envelope, thus, is sensitive and susceptible to host immune defenses, which can affectively block the delivery of the recombinant genome.

One method for increasing the efficacy of nucleic acid delivery is to conjugate the administered agent with a polymer, such as polyethylene glycol (PEG). PEG provides some increase in effective size of the agent and it can provide some steric protection from enzymatic degradation thereby improving the circulation time and hence bioavailability. PEG can also provide hydrophilicity to lipophilic compounds. PEG is a linear polymer of ethylene glycol, synthesized by a process of condensation at high pressure that leads to large heterogeneity in molecular weight. Also, since it is a linear polymer, the steric protection due to PEG is limited as compared to polymers that have branching and hence can occupy a larger space. A major shortcoming of PEG is that it is not very amenable to derivatization except at the two terminals. Difficulties in introducing other functional molecules helpful for nucleic acid delivery to specific tissues are well known.

Other efforts have focused on administering a combination of plasmids, one conveying the genome of a virus with a different gene for its outer envelop protein taken from a different virus specific for a different species host (this change makes the virus unable to bind and infect human cells); and the other conveying the receptor needed by the new envelop protein (Matano et al., Vaccine 2000 18, 3310-8). These processes are cumbersome, as well as expensive. Accordingly, there is a need for a gene delivery vehicle that is capable of effectively delivering an exogenous gene to a targeted cell, yet does not elicit a humoral or cellular immune response upon repeated interaction with the cellular environment.

Another drawback to administering live, attenuated viruses is the considerable safety risk they pose. While efforts have been applied to control viral replication mechanism, certain levels of replication are needed to meet desirable efficacy levels. This dilemma is apparent in HIV vaccines, for failure of controlling replication can result in the transmission of AIDS.

Non-viral delivery systems have been developed to overcome the safety problems associated with live vectors. Although such non-viral systems generally are permissive of repeated administration and often are able to incorporate a wide variety of nucleic acid compositions; they often are limited by low efficiency and a very short persistence. Also, while non-viral vectors do not suffer from the same safety problems as those of viral vectors, they do have their own toxicity problems. For example, two of the most widely used polycations for gene delivery, poly-L-lysine and polyethyleneimine, are limited in their use in mammals by significant systemic and organ toxicity, including severe adverse reactions in liver and lung tissues. These toxicity problems need to be managed by chemical modifications that address the specific toxicity problems.

First generation non-viral vector systems are simple cationic complexes based on two classes of molecule, polymers and lipids, both cationic in nature. Most commonly used cationic polymers for gene delivery are poly-(L-lysine)(PLL), and poly-(ethylineimine) (PEI). PEI is also proposed to have endosome buffering activity that leads to endosome disruption. These polycations bind and condense DNA into small particles. This enables the uptake of the particles and also protects the DNA from enzymatic degradation.

There is, accordingly, a need for finding gene delivery, systems that: (i) are less toxic than conventionally used vectors, (ii) prolong persistence in vivo and (iii) provide for selective expression in target tissues giving therapeutically effective levels of the therapeutic agent.

### SUMMARY OF TE INVENTION

One embodiment of the invention provides compositions for nucleic acid delivery comprising HPMA conjugated to a polyamine.

In a preferred embodiment, the polyamine is selected from the group consisting of: spermine, spermidine and their analogues, and DFMO.

In another embodiment, the composition further comprises a targeting ligand.

In yet another embodiment, the composition further comprises one or more ligand binding domains.

In a preferred embodiment, the targeting ligands are selected from the group consisting of: vascular endothelial growth factors, somatostatin and somatostatin analogs, transferrin, melanotropin, ApoE and ApoE peptides, von Willebrand's factor and von Willebrand's factor peptides, adenoviral fiber protein and adenoviral fiber protein peptides, PD1 and PD1 peptides, EGF and EGF peptides, RGD peptides, CCK peptides, antibody and antibody fragments, folate, pyridoxyl and sialyl-LewisX and chemical analogs and DNA and RNA aptamers.

In a further preferred embodiment, the antibody fragment is selected from the group consisting of: Fab′, F(ab′)2, Fab, Facb, Fd, Fv, and scFv.

Another embodiment of the invention provides a method for delivering a nucleic acid to an in vivo system, comprising administering to a subject an effective amount of a therapeutic nucleic acid bound to a polyamine that is conjugated to HPMA.

Another embodiment of the invention provides methods for delivering one or more nucleic acids to a cell utilizing compositions comprising HPMA conjugated to a polyamine

Another embodiment of the invention provides methods for delivering one or more nucleic acids to a cell utilizing compositions comprising HPMA conjugated to a polyamine and targeting ligands.

A further embodiment provides methods of administering a composition comprising HPMA conjugated to a polyamine, wherein a nucleic acid is bound to the polyamine, to a subject that leads to a therapeutic effect.

A further embodiment provides methods of administering a composition comprising HPMA conjugated to a polyamine and targeting ligand, wherein a nucleic acid is bound to the polyamine, to a subject that leads to a therapeutic effect.

A further embodiment provides a method of administering a therapeutic agent comprising HPMA conjugated to a polyamine, wherein a nucleic acid is bound to the polyamine, and wherein the compositions may be administered in repeated doses.

A further embodiment provides a method of administering a therapeutic agent comprising HPMA conjugated to a polyamine and targeting ligand, wherein a nucleic acid is bound to the polyamine, and wherein the compositions may be administered in repeated doses.

Another embodiment provides a method for inhibiting cellular proliferation, comprising administering an effective amount of a therapeutic nucleic acid via a polyamine conjugated to BMA.

Another embodiment provides a method for inhibiting cellular proliferation, comprising administering an effective amount of a therapeutic nucleic acid via a polyamine and targeting ligand conjugated to HPMA.

A further embodiment provides a method of vaccination comprising the steps of administering to a subject an effective amount of a therapeutic nucleic acid bound to a polyamine that is conjugated to HPMA and administering subsequent doses of the same therapeutic nucleic acid at intervals designed to optimize immune response.

### DETAILED DESCRIPTION OF THE PREFERRED EMBODIMENTS

The term, “nucleic acid” refers to any variety of DNA or RNA molecule, including but not limited to, mRNA, double stranded RNA, interfering RNA, cDNA, single stranded DNA, double stranded DNA, plasmid DNA, viral DNA, sense or antisense molecules, and fragments of any of these varieties. The interfering RNA may be an interfering double stranded RNA (RNAi) or an siRNA, or a polynucleotide encoding a double stranded RNA. Examples of suitable double stranded RNA molecules and vectors encoding such molecules are described in, for example, U.S. Pat. No. 6,506,559, which is hereby incorporated by reference in its entirety.

The term “gene delivery agent” refers to HPMA conjugated to a polyamine. A gene delivery agent of the invention preferably is conjugated to a ligand or tissue targeting domain while retaining (or substantially retaining) its desired characteristics.

Compounds of the Invention

As used herein, the term “HPMA” means the compound N-2-hydroxypropyl methacrylamide, which is a hydrophilic polymer represented by the following structure:

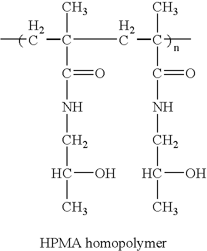

The term “polyamine” as used herein refers to DFMO, spermine NH₂(CH₂)₃NH(CH₂)₄NH(CH₂)₃NH₂, spermidine NH₂(CH₂)₄NH(CH₂)₃NH₂, and synthetic spermine analogs having a formula R₁—NH—(CH₂)_{w}—NH—(CH₂)ₓ—NH—(CH₂)_{y}—NH—(CH₂)_{z}—NH—R₂, wherein R₁ and R₂ are hydrocarbon chains having 1 to 5 carbons and w, x, y and z are integers of 1 to 10. More preferably, R₁ and R₂ are hydrocarbon chains having 2 carbons and w, x, y and z are integers of 3 or 4. Additionally, substitutions of certain hydrogens and carbons with other atoms or molecules may be undertaken without departing from the scope of the present invention. These compounds are therapeutic polyamines, useful as cancer chemotherapeutic agents.

The hydrocarbon chains may be an alkyl group, an alkenyl group, or an alkynyl group.

The term “alkyl”, alone or in combination with any other term, refers to a straight-chain or branch-chain saturated aliphatic hydrocarbon radical containing the specified number of carbon atoms, or where no number is specified, preferably from 1 to about 15 and more preferably from 1 to about 10 carbon atoms. Examples of alkyl radicals include, but are not limited to, methyl, ethyl, n-propyl, isopropyl, n-butyl, isobutyl, sec-butyl, tert-butyl, pentyl, isoamyl, n-hexyl and the like.

The term “alkenyl”, alone or in combination with any other term, refers to a straight-chain or branched-chain mono- or poly-unsaturated aliphatic hydrocarbon radical containing the specified number of carbon atoms, or where no number is specified, preferably from 2-10 carbon atoms and more preferably, from 2-6 carbon atoms. Examples of alkenyl radicals include, but are not limited to, ethenyl, E- and Z-propenyl, isopropenyl, E- and Z-butenyl, E- and Z-isobutenyl, E- and Z-pentenyl, E- and Z-hexenyl, E,E-, E,Z-, Z,E- and Z,Z-hexadienyl and the like.

The term “alkynyl,” alone or in combination with any other term, refers to a straight-chain or branched-chain hydrocarbon radical having one or more triple bonds containing the specified number of carbon atoms, or where no number is specified, preferably from 2 to about 10 carbon atoms. Examples of alkynyl radicals include, but are not limited to, ethynyl, propynyl, propargyl, butynyl, pentynyl and the like.

In a preferred embodiment, the targeting ligand of the instant invention may comprise, for example, a targeting ligand or moiety for targeting specific cells and tissues. In another preferred embodiment, it may contain a fusogenic moiety for facilitating entry of an agent, preferably a nucleic acid, into a cell. In yet another preferred embodiment, it may contain a nuclear targeting moiety for targeting specific cells and tissues.

A targeting ligand enhances binding of the polymer to target tissue or cells and permits highly specific interaction of the polymers with the target tissue or cell. In one embodiment, the polymer will include a ligand effective for ligand-specific binding to a receptor molecule on a target tissue and cell surface (Woodle et al., Small molecule ligands for targeting long circulating liposomes, in Long Circulating Liposomes: Old drugs, new Therapeutics, Woodle and Storm eds., Springer, 1998, p 287-295).

The polymer may include two or more targeting moieties, depending on the cell type that is to be targeted. Use of multiple targeting moieties can provide additional selectivity in cell targeting, and also can contribute to higher affinity and/or avidity of binding of the polymer to the target cell. When more than one targeting moiety is present on the polymer, the relative molar ratio of the targeting moieties may be varied to provide optimal targeting efficiency. Methods for optimizing cell binding and selectivity in this fashion are known in the art. The skilled artisan also will recognize that assays for measuring cell selectivity and affinity and efficiency of binding are known in the art and can be used to optimize the nature and quantity of the targeting ligand(s).

Suitable ligands include, but are not limited to: vascular endothelial cell growth factor for targeting endothelial cells: FGF2 for targeting vascular lesions and tumors; somatostatin peptides for targeting tumors; transferrin for targeting tumors; melanotropin (alpha MSH) peptides for tumor targeting; ApoE and peptides for LDL receptor targeting; von Willebrand's Factor and peptides for targeting exposed collagen; Adenoviral fiber protein and peptides for targeting Coxsackie-adenoviral receptor (CAR) expressing cells; PD1 and peptides for targeting Neuropilin 1; EGF and peptides for targeting EGF receptor expressing cells; and RGD peptides for targeting integrin expressing cells and DNA and RNA aptamers.

Other examples include (i) folate, where the polymer is intended for treating tumor cells having cell-surface folate receptors, (ii) pyridoxyl, where the polymer is intended for treating virus-infected CD4+ lymphocytes, or (iii) sialyl-Lewis, where the polymer is intended for treating a region of inflammation. Other peptide ligands may be identified using methods such as phage display (F. Bartoli et al., Isolation of peptide ligands for tissue-specific cell surface receptors, in Vector Targeting Strategies for Therapeutic Gene Delivery (Abstracts form Cold Spring Harbor Laboratory 1999 meeting), 1999, p4) and microbial display (Georgiou et al., Ultra High Affinity Antibodies from Libraries Displayed on the Surface of Microorganisms and Screened by FACS, in Vector Targeting Strategies for Therapeutic Gene Delivery (Abstracts form Cold Spring Harbor Laboratory 1999 meeting), 1999, p 3.).

In one embodiment, the targeting ligand may be somatostatin or a somatostatin analog. Somatostatin has the sequence AGCLNFFWKTFTSC, and contains a disulfide bridge between the cysteine residues. Many somatostatin analogs that bind to the somatostatin receptor are known in the art and are suitable for use in the present invention, such as those described, for example, in U.S. Pat. No. 5,776,894, which is incorporated herein by reference in its' entirety. Particular somatostatin analogs that are useful in the present invention are analogs having the general structure F*CY-(DW)KTCT, where DW is D-tryptophan and F* indicates, that the phenylalanine residue may have either the D- or L-absolute configuration. As in somatostatin itself, these compounds are cyclic due to a disulfide bond between the cysteine residues. Advantageously, these analogs may be derivatized at the free amino group of the phenylalanine residue, for example with a polycationic moiety such as a chain of lysine residues. The skilled artisan will recognize that other somatostatin analogs that are known in the art may advantageously be used in the invention.

Furthermore, methods have been developed to create novel peptide sequences that elicit strong and selective binding for target tissues and cells such as “DNA Shuffling” (W. P. C. Stremmer, Directed Evolution of Enzymes and Pathways by DNA Shuffling, in Vector Targeting Strategies for Therapeutic Gene Delivery (Abstracts form Cold Spring Harbor Laboratory 1999 meeting), 1999, p.5.) and these novel sequence peptides are suitable ligands for the invention. Other chemical forms for ligands are suitable for the invention such as natural carbohydrates which exist in numerous forms and are a commonly-used ligand by cells (Kraling et al., Am. J. Path. 150:1307 (1997) as well as novel chemical species, some of which may be analogues of natural ligands such as D-amino acids and peptidomimetics and others which are identified through medicinal chemistry techniques such as combinatorial chemistry (P. D. Kassner et al., Ligand Identification via Expression (LIVE): Direct selection of Targeting Ligands from Combinatorial Libraries, in Vector Targeting Strategies for Therapeutic Gene Delivery (Abstracts form Cold Spring Harbor Laboratory 1999 meeting), 1999, p8.).

The targeting moiety provides tissue- and cell-specific binding. The ligands may be covalently attached to the polymer so that exposure is adequate for tissue and cell binding. For example, a peptide ligand can be covalently coupled to a polymer such as polyoxazoline.

The number of targeting molecules present on the outer layer will vary, depending on factors such as the avidity of the ligand-receptor interaction, the relative abundance of the receptor on the target tissue and cell surface, and the relative abundance of the target tissue and cell. Nevertheless, a targeting molecule coupled with of each polymer usually provides suitable enhancement of cell targeting.

The presence of the targeting moiety leads to the desired enhancement of binding to target tissue and cells. An appropriate assay for such binding may be ELISA plate assays, cell culture expression assays, or any other binding assays.

The fusogenic moiety promotes fusion of the polymer to the cell membrane of the target cell, facilitating entry of the polymer and therapeutic agents into the cell. In one embodiment, the fusogenic moiety comprises a fusion-promoting element. Such elements interact with cell membranes or endosome membranes in a manner that allows transmembrane movement of large molecules or particles, or disrupts the membranes such that the aqueous phases that are separated by the membranes may freely mix. Examples of suitable fusogenic moieties include, but are not limited to membrane surfactant peptides, e.g. viral fusion proteins such as hemagglutinin (HA) of influenza virus, or peptides derived from toxins such as PE and ricin. Other examples include sequences that permit cellular trafficking such as HIV TAT protein and antennapedia or those derived from numerous other species, or synthetic polymers that exhibit pH sensitive properties such as poly(ethylacrylic acid)(Lackey et al., Proc. Int. Symp. Control. Rel. Bioact. Mater. 1999, 26, #6245), N-isopropylacrylamide methacrylic acid copolymers (Meyer et al., FEBS Lett. 421:61 (1999)), or poly(amidoamine)s, (Richardson et al., Proc. Int. Symp. Control. Rel. Bioact. Mater. 1999, 26, #251), and lipidic agents that are released into the aqueous phase upon binding to the target cell or endosome. Suitable membrane surfactant peptides include an influenza hemagglutinin or a viral fusogenic peptide such as the Moloney murine leukemia virus (“MoMuLV” or MLV) envelope (env) protein or vesicular stroma virus (VSV) G-protein. The membrane-proximal cytoplasmic domain of the MoMuLV env protein may be used. This domain is conserved among a variety viruses and contains a membrane-induced α-helix.

Suitable viral fusogenic peptides for the instant invention may include a fusion peptide from a viral envelope protein ectodomain, a membrane-destabilizing peptide of a viral envelope protein membrane-proximal domain, hydrophobic domain peptide segments of so called viral “fusion” proteins, and an amphiphilic-region containing peptide. Suitable amphiphilic region containing peptides include, but are not limited to: melittin, the magainins, fusion segments from H. influenza hemagglutinin (HA) protein, HIV segment I from the cytoplasmic tail of HIV1 gp41, and amphiphilic segments from viral env membrane proteins including those from avian leukosis virus (ALV), bovine leukemia virus (BLV), equine infectious anemia (EIA), feline immunodeficiency virus (FM), hepatitis virus, herpes simplex virus (HSV) glycoprotein H, human respiratory syncytia virus (hRSV), Mason-Pfizer monkey virus (MPMV), Rous sarcoma virus (RSV), parainfluenza virus (PINF), spleen necrosis virus (SNV), and vesicular stomatitis virus (VSV). Other suitable peptides include microbial and reptilian cytotoxic peptides. The specific peptides or other molecules having greatest utility can be identified using four kinds of assays: 1) ability to disrupt and induce leakage of aqueous markers from liposomes composed of cell membrane lipids or fragments of cell membranes, 2) ability to induce fusion of liposomes composed of cell membrane lipids or fragments of cell membranes, 3) ability to induce cytoplasmic release of particles added to cells in tissue culture, and 4) ability to enhance plasmid expression by particles in vivo tissues when administered locally or systemically.

The fusogenic moiety also may be comprised of a polymer, including peptides and synthetic polymers. In one embodiment, the peptide polymer comprises synthetic peptides containing amphipathic aminoacid sequences such as the “GALA” and “KALA” peptides (Wyman T B, Nicol F, Zelphati O, Scoria P V, Plank C, Szoka F C Jr, Biochemistry 1997, 36:3008-3017; Subbarao N K, Parente R A, Szoka F C Jr, Nadasdi L, Pongracz K. Biochemistry 1987 26:2964-2972 or Wyman supra, Subbarao supra ). Other peptides include non-natural aminoacids, including D aminoacids and chemical analogues such as peptoids, imidazole-containing polymers. Suitable polymers include molecules containing amino or imidazole moieties with intermittent carboxylic acid functionalities such as ones that form “salt-bridges,” either internally or externally, including forms where the bridging is pH sensitive. Other polymers can be used including ones having disulfide bridges either internally or between polymers such that the disulfide bridges block fusogenicity and then bridges are cleaved within the tissue or intracellular compartment so that the fusogenic properties are expressed at those desired sites. For example, a polymer that forms weak electrostatic interactions with a positively charged fusogenic polymer that neutralizes the positive charge could be held in place with disulfide bridges between the two molecules and these disulfides cleaved within an endosome so that the two molecules dissociate releasing the positive charge and fusogenic activity. Another form of this type of fusogenic agent has the two properties localized onto different segments of the same molecule and thus the bridge is intramolecular so that its dissociation results in a structural change in the molecule. Yet another form of this type of fusogenic agent has a pH sensitive bridge.

The fusogenic moiety also may comprise a membrane surfactant polymer-lipid conjugate. Suitable conjugates include Thesit™, Brij 58™, Brij 78™, Tween 80™, Tween 20™, C₁₂E₈, C₁₄E₈, C₁₆E₈ (CₙEₙ,=hydrocarbon poly(ethylene glycol)ether where C represents hydrocarbon of carbon length N and E represents poly(ethylene glycol) of degree of polymerization N), Chol-PEG 900, analogues containing polyoxazoline or other hydrophilic polymers substituted for the PEG, and analogues having fluorocarbons substituted for the hydrocarbon. Advantageously, the polymer will be either biodegradable or of sufficiently small molecular weight that it can be excreted without metabolism. The skilled artisan will recognize that other fusogenic moieties also may be used without departing from the spirit of the invention.

A major barrier to efficient transcription and consequent expression of an exogenous nucleic acid moiety is the requirement that the nucleic acid enter the nucleus of the target cell. Advantageously, when the intended biological target of a nucleic acid is the nucleus, the nucleic targeting moiety of the invention is “nuclear targeted,” that is, it contains one or more molecules that facilitate entry of the nucleic, acid through the nuclear membrane into the nucleus of the host cell. Such nuclear targeting may be achieved by incorporating a nuclear membrane transport peptide, or nuclear localization signal (“NLS”) peptide, or small molecule that provides the same NLS function, into the core complex. Suitable peptides are described in, for example, U.S. Pat. Nos. 5,795,587 and 5,670,347 and in patent application WO 9858955, which are hereby incorporated by reference in their entirety, and in Aronsohn et al., J. Drug Targeting 1:163 (1997); Zanta et al., Proc. Nat'l Acad. Sci. USA 96:91-96 (1999); Ciolina et al., Targeting of Plasmid DNA to Importin alpha by Chemical coupling with Nuclear Localization Signal Peptides, in Vector Targeting Strategies for Therapeutic Gene Delivery (Abstracts from Cold Spring Harbor Laboratory 1999 meeting), 1999, p 20; Saphire et al., J. Biol. Chem; 273:29764 (1999). A nuclear targeting peptide may be a nuclear localization signal peptide or nuclear membrane transport peptide and it may be comprised of natural amino acids or non-natural amino acids including D amino acids and chemical analogues such as peptoids. The NLS may be comprised of amino acids or their analogues in a natural sequence or in reverse sequence. Another embodiment provides a steroid receptor-binding NLS moiety that activates nuclear transport of the receptor from the cytoplasm, wherein this transport carries the nucleic acid with the receptor into the nucleus.

In another embodiment, the NLS is coupled to the polymer in such a manner that the polymer is directed to the cell nucleus where it permits entry of a nucleic acid into the nucleus.

In another embodiment, incorporation of the NLS moiety into the polymer occurs through association with the nucleic acid, and this association is retained within the cytoplasm. This minimizes loss of the NLS function due to dissociation with the nucleic acid and ensures that a high level of the nucleic acid is delivered to the nucleus. Furthermore, the association with the nucleic acid does not inhibit the intended biological activity within the nucleus once the nucleic acid is delivered.

In yet another embodiment, the intended target of the biological activity of the nucleic acid is the cytoplasm or an organelle in the cytoplasm such as ribosomes, the golgi apparatus, or the endoplasmic reticulum. In this embodiment, a localization signal is included in the polymer anchored to it so that it provides direction of the nucleic acid to the intended site where the nucleic acid exerts its activity. Signal peptides that can achieve such targeting are known in the art.

Methods of Making HPMAA-polyamine Conjugates

The synthesis of the HPMA-polyamine conjugates as well as their individual components is discussed thoroughly in the Examples.

An HPMA-polyamine conjugate (optionally attached to other moieties) can be used in a variety of ways to bring about a therapeutic effect. An HPMA-polyamine conjugate is particularly suitable for delivering an effective amount of a therapeutic agent to an in vivo system over an extended period of time due to the presence of biodegradable side chains of HPMA which are cleavable specifically by the enzymes in the lysosomal compartment. This finding is significant, given the limitations of state of the art delivery compositions. For delivery of genes, HPMA copolymer DFMO conjugates can be synthesized containing non-degradable glycylglycine (GG) spacers between the polymer and the polyamine. Complexes of these cationic polymer conjugates with negatively charged DNA will allow the delivery of nucleic acids to a subject in a timed-dose manner, wherein greater concentrations of HPMA-polyamine conjugate may result in controlled release of the nucleic acid to the subject. As a result, the gene delivery vehicles of the invention can be useful in a number of therapeutic applications, including: therapeutic vaccines, preventative vaccines, treatment of inflammatory disorders and many types of malignancies, as well as any other regimen involving repeated administration or expression of a therapeutic agent including nucleic acid molecules.

Preferably, a nucleic acid delivery vehicle for use in the present invention has the ability to deliver a therapeutic nucleic acid molecule to an in vitro system, e.g., a mammalian system, without stimulating an immune response that causes substantial and/or premature clearance of the gene delivery vehicle from the in vivo system.

The invention contemplates using any conventionally available ligand domain as a targeting means, provided that it does not inhibit delivery and expression of the therapeutic nucleic acid.

Enhanced Delivery of a Nucleic Acid Therapeutic Agent

The invention also contemplates enhanced delivery of a nucleic acid therapeutic agent by employing oral administration. Enhanced delivery can result from protection of the agent by the polymer and binding to target tissues and cells in the gastrointestinal tract.

Therapeutic Methods

The present invention provides methods of administering one or more therapeutic nucleic acids to a subject, using a vehicle comprised of HPMA conjugated to a polyamine, to bring about a therapeutic benefit to the subject. As used herein, a “therapeutic nucleic acid” is any gene delivery agent that can confer a therapeutic benefit to a subject. The subject preferably is mammalian such as a mouse, and more preferably is a human being.

Delivery vehicles for use in the present invention can be used to stimulate an immune response, which may be protective or therapeutic. Accordingly, the delivery vehicles can be used to vaccinate a subject against an antigen.

In this sense, the invention provides methods vaccinating or enhancing a physiological response against a pathogen in a subject. This methodology can entail administering to the subject a first, or priming, dosage of a therapeutic nucleic acid molecule that encodes a therapeutic polypeptide, followed by administering to the subject one or more booster dosages of the nucleic acid molecule.

The administration regimen can vary, depending on, for example, (i) the subject to whom the therapeutic nucleic acid molecule is administered, and (ii) the pathogen that is involved. For instance, a booster dosage of a therapeutic nucleic acid molecule may be administered about two weeks after priming, followed by successive booster dosages, which can occur between intervals of constant or increasing duration. It is desirable to administer therapeutic nucleic acid molecules at a periodicity that is appropriate according to the subject's immune response.

In the preceding administration steps, the administered nucleic acid molecule is comprised within a gene delivery vehicle of the invention. Preferably, expression of the therapeutic nucleic acid molecule in the foregoing steps elicits a humoral and/or cellular response in the subject, causing the subject to exhibit a degree of immunity against the pathogen that is greater than before the therapeutic method is carried out.

The antigen against which the subject exhibits an increased immunity can be the antigen encoded by the therapeutic nucleic acid molecule. Alternatively, the polypeptide against which the subject exhibits an increased immunity is distinct from, or in addition to, the polypeptide expressed by the administered nucleic acid molecule. In the latter approach, for instance, the polypeptide encoded by the therapeutic nucleic acid can act to enhance an immune response against another antigen, e.g., a component of a tumor.

The route of administration may vary, depending on the therapeutic application (e.g., preventative or therapeutic vaccine) and the type of disorder to be treated. The gene delivery vehicle may be administered by injection into the skin or muscle; intravenously; directly to the portal vein, hepatic vein or bile duct; locally to a tumor or to a joint.

An administered therapeutic nucleic acid molecule also may induce an immune response. A response can be achieved to intracellular infectious agents including, for example, tuberculosis, Lyme disease, and others. A response can be achieved by expression of antigen, expression of cytokines, or their combination. The invention also provides for expression of HIV antigens and induction of both a protective and a therapeutic immune response for preventing and treating HIV, respectively.

The invention additionally provides for the expression of antigens, which elicit a humoral and/or a cellular immune response. This heightened immune response can provide protection from a challenge with infectious agents characterized as having the antigen. Preferably, the invention utilizes an adenoviral genomic nucleic acid that (i) expresses an antigen under-control of a promoter and (ii) targets an APC.

In one embodiment, the therapeutic nucleic acid encodes a cytokine, which may be expressed with or without an antigen. A cytokine acts to recruit an immune response, which can enhance an immune response to an expressed antigen. Accordingly, cytokine expression can be obtained whereby APCs and other immune response cells are recruited to the vicinity of tumor cells, in which case there is no requirement for co-expression of an antigen by the gene delivery vehicle. Yet, in another embodiment, one or more antigens and cytokines can be co-expressed.

Accordingly, the invention contemplates the use of immunostimulatory cytokines, as well as protein analogues exhibiting biological activity similar to an immunostimulatory cytokine, to vaccinate a subject. Suitable cytokines for use in enhancing an immune response include GM-CSF, IL-1, IL-2, IL-12, IL-15, interferons, B-40, B-7, tumor necrosis factor (TNF) and others. The invention also contemplates utilizing genes that down-regulate immunosuppressants cytokines.

The invention also provides for expression of “recruitment cytokines” at tumors. Expression of cytokines at tumors giving recruitment of immune response cells can initiate a cellular immune response at the tumor site giving recognition and killing of tumor cells at the site of expression and at distal tumor sites. A preferred embodiment of the invention is comprised of an adenoviral genomic nucleic acid, the nucleic acid exhibiting expression of GM-CSF under a tumor-preferential promoter, further comprised of nucleic acid exhibiting tumor-conditional replication to form adenoviral vector particles exhibiting tumor-conditional replication, and yet further comprised by synthetic vector compositions targeting delivery to tumor lesions. Another preferred embodiment of the invention utilizes an adenoviral genomic nucleic acid encoding a cytokine (e.g., GM-CSF) under regulation of a tumor-conditional promoter. This feature would result in enhanced cytokine expression at the site of a tumor. In this embodiment, the adenoviral genomic nucleic acid preferably is administered in conjunction with electroporation to tumor lesions.

An HPMA-polyamine conjugate also may be used to deliver an agent that treats a disorder characterized by inflammation. In one approach, one or more therapeutic agents are administered to a subject suffering from a disorder characterized by inflammation, in order to suppress or retard an immune response. Treatable disorders include rheumatoid arthritis, psoriasis, gout and inflammatory bowel disorders.

Suitable therapeutic agent for use in treating inflammation include inflammation inhibitory cytokines, such as: IL-1RA, soluble TNF receptor, and soluble Fas ligand.

The route and site of administration will vary, depending on the disorder and the location of inflammation. The gene delivery agent can be administered into a joint; administration thereto can be in conjunction with electroporation.

Pharmaceutical Compositions

The amount of active ingredient that may be combined with the carrier materials to produce a single dosage form will vary depending upon the host treated and the particular mode of administration.

The dosage regimen for treating a disease condition with the compounds and/or compositions of this invention is selected in accordance with a variety of factors, including the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized and whether the compound is administered as part of a drug combination. Thus, the dosage regimen actually employed may vary widely and therefore may deviate from the preferred dosage regimen set forth above.

The compounds of the present invention may be administered orally, parenterally, by inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols which are solid at ordinary temperatures but liquid at the rectal temperature and will therefore melt in the rectum and release the drug

Solid dosage forms for oral administration may include capsules, tablets, pills, powders, and granules. In such solid dosage forms, the active compound may be admixed with at least one inert diluent such as sucrose lactose or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally by prepared with enteric coatings

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

While the compounds of the invention can be administered as the sole active pharmaceutical agent, they can also be used in combination with one or more therapeutic agents, such as immunomodulators, antiviral agents or antiinfective agents.

The foregoing is merely illustrative of the invention and is not intended to limit the invention to the disclosed compounds. Variations and changes which are obvious to one skilled in the art are intended to be within the scope and nature of the invention which are defined in the appended claims. From the foregoing description, one skilled in the art can easily ascertain the essential characteristics of this invention, and without departing from the spirit and scope thereof, can make various changes and modifications of the invention to adapt it to various usages and conditions.

All references listed herein are incorporated herein by reference in their entireties, including the priority document, U.S. Provisional Application No. 60/352,883, filed Feb. 1, 2002, which is incorporated herein by in its entirety.

### EXAMPLES

### Example A1

### Synthesis of HPMA Polyamine Conjugates for Gene Delivery

HPMA polyamine conjugates will be synthesized by the polymerization, of HPMA monomer and an activated MA-GFLG comonomer at different molar ratios followed by reaction with the amino function of the polyamine molecule.

Synthesis of Polymeric Co-Monomers

Synthesis of the reactive comonomer HPMA was as previously described (Kopecek and Bazilova, 1973). The synthesis of the MA-GFLG-ONp monomer was a modified multistep procedure (Kopecek et al., 1985). First MA-Gly-Phe and Leu-Gly-OMe.HCl were synthesized separately. Subsequently the two dipeptides were coupled to yield MA-GFLG-OMe. The methyl-group was removed with base giving MA-GFLG-OH and to this compound the reactive group p-nitrophenol was attached by esterification.

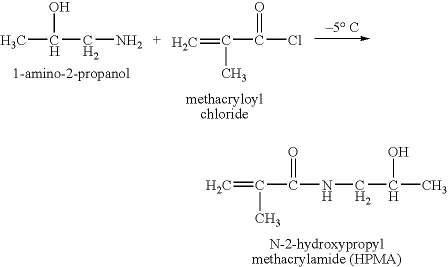

Synthesis of HPMA

To a solution of 1-amino-2-propanol (65.6 ml, 0.84 mol) in 250 ml of acetonitrile, freshly distilled methacryloyl chloride (MACl) (41 ml, 0.42 mol) in 20 ml of acetonitrile was added dropwise under vigorous stirring and cooling to −5° C. A small amount of inhibitor, tertiary octyl pyrocatechine was added. The reaction mixture was stirred for an additional 30 min at room temperature. 1-amino-2-propanol hydrochloride formed as a byproduct was precipitated and filtered off. The filtrate was cooled to −70° C. and the HPMA precipitated. After equilibrating to room temperature the product was filtered off and washed with pre-cooled acetonitrile. Recrystallization was from acetone and the pure product was isolated.

Synthesis of MA-GF-OH

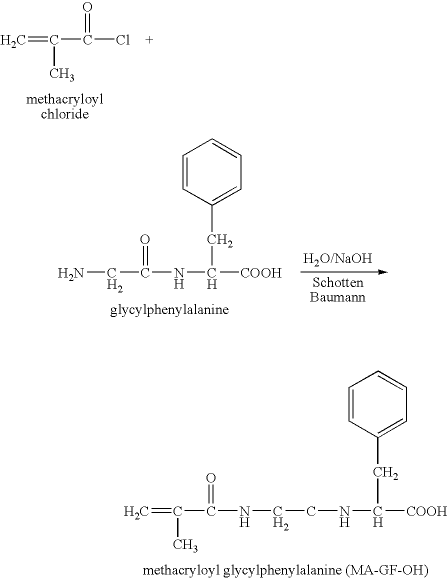

Glycylphenylalanine (Gly-Phe, 5.0 g, 22.5 mmol) was dissolved in 5.6 ml of 4N NaOH (22.5 mmol) and cooled to 0° C. Freshly distilled MACl (3.5 g, 34 mmol) in 10 ml of dichloromethane was added dropwise. A small amount of inhibitor, tertiary octyl pyrocatechine was added to prevent polymerization of the monomer. Simultaneously but with a slight delay, 8.4 ml (34 mmol) of 4N NaOH was added dropwise to the reaction mixture. After addition of MACl and NaOH the reaction mixture was warmed up to room temperature and allowed to react for one hour. The pH was maintained at around 6-7. The dichloromethane layer was separated from the water layer, washed with 2 ml of water and discarded. The aqueous layer together with the washings was mixed with 40 ml of EtOAc. Under vigorous stirring and cooling, HCl (36.5%) was added slowly until the pH reached at 2-3. The organic layer was separated and the aqueous layer was extracted three times with EtOAc (3×20 ml). The extracted layers was dried over anhydrous sodium sulfate overnight. The dried solution was filtered and washed with EtOAc. The EtOAc was, removed by rotoevaporating to obtain the product as a white powder. Recrystallization was from EtOAc.

Synthesis of LG-OMe.HCl

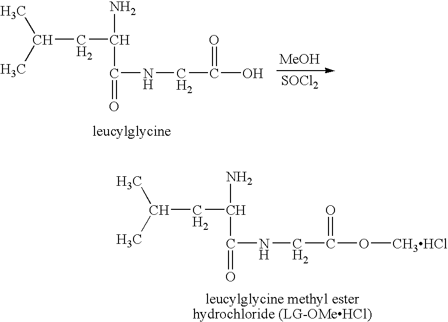

Leucylglycine (Leu-Gly, 4.0 g 21 mmol) was dissolved in 35 ml of methanol and cooled to −5° C. 2 ml (26 mmol) of SOCl₂ was added dropwise under stirring. After equilibrating to room temperature the mixture was refluxed for three hours. The solvent was evaporated to dryness and the residue was dissolved in methanol and evaporated to remove HCl and SOCl₂. The residue was dissolved in benzene and evaporated to obtain a white amorphous solid. This was used in subsequent steps without purification.

Synthesis of MA-GFLG-OMe

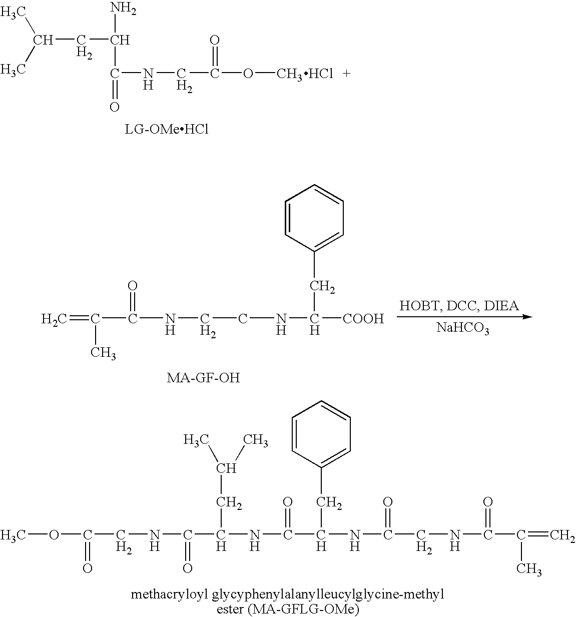

To a solution of Leu-Gly-OMe.HCl (5.0 g, 21 mmol) in 40 ml of DMF, was added 4.0 g of HOBT (25 mmol), 4.0 ml of DIEA (25 mmol) and the MA-Gly-Phe (6.0 g, 20.7 mmol). The reaction mixture was stirred and cooled to −10° C. 5.2 g of DCC (25 mmol) in 20 nm of DMF was added dropwise within five minutes. The solution was stirred for two hours at 0° C. and then for 24 hours at room temperature. After overnight stirring the precipitated byproduct dicyclohexyl urea (DCU) was filtered off. The filtrate was roto-evaporated to remove the DMF completely. The residue was mixed with 40 ml of 5% NaHCO₃ solution and extracted with EtOAc three times (3×80 ml). The extract was washed with 40 ml of 5% citric acid solution, 40 ml of 5% NaHCO₃ solution and 3×40 ml of saturated brine and dried over anhydrous sodium sulfate for two hours. After filtering off the drying agent and addition of a small amount of inhibitor tertiary octyl pyrocatechine the filtrate was concentrated under vacuum to obtain the product. Recrystallization was done from EtOAc.

Synthesis of MA-GFLG-OH

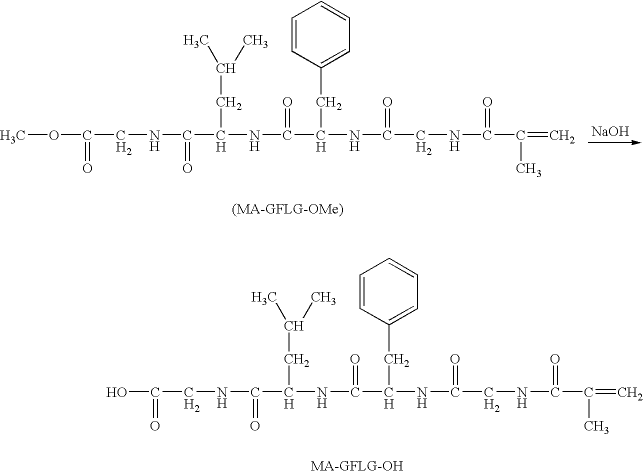

To a solution of MA-GFLG-OMe (6.9 g, 14.5 mmol) in 80 ml of methanol and cooled to 0° C., excess of 1N NaOH (18 ml, 18 mmol) was added dropwise under stirring. After addition of a small amount of inhibitor (t-octyl pyrocatechine) the reaction mixture was stirred for one and a half hours at 0° C. and then for two hours at room temperature. The reaction mixture was concentrated under vacuum to remove methanol. 160 ml of distilled water was added and the mixture was acidified with concentrated citric acid to pH 2.0. The free acid was extracted with 4×200 ml of EtOAc, washed with saturated brine and dried over anhydrous sodium sulfate overnight. After evaporation of the solvent under vacuum the tetrapeptide product was re-crystallized from EtOAc.

Synthesis of MA-GFLG-ONp

To a solution of MA-GFLG-OH (4.7 g, 10 mmol) in 80 ml of DMF a solution of 1.67 g of p-nitrophenol (12 mmol) in 20 ml of DMF was added under stirring and cooling to −10° C. followed by a solution of 2.5 g of DCC (12 mmol) in 8 ml of DMF. The reaction mixture was stirred for six hours at −10° C. and then overnight at 4° C. The precipitated byproduct DCU was filtered off and the DMF was removed by rotary evaporation. The residue was dissolved in EtOAc and the remaining byproduct was filtered off. EtOAc was evaporated to dryness. The product was soaked in ether to remove excess p-nitrophenol. This procedure was repeated several times and the purity of the product was checked by calculating the extinction coefficient in DMSO.

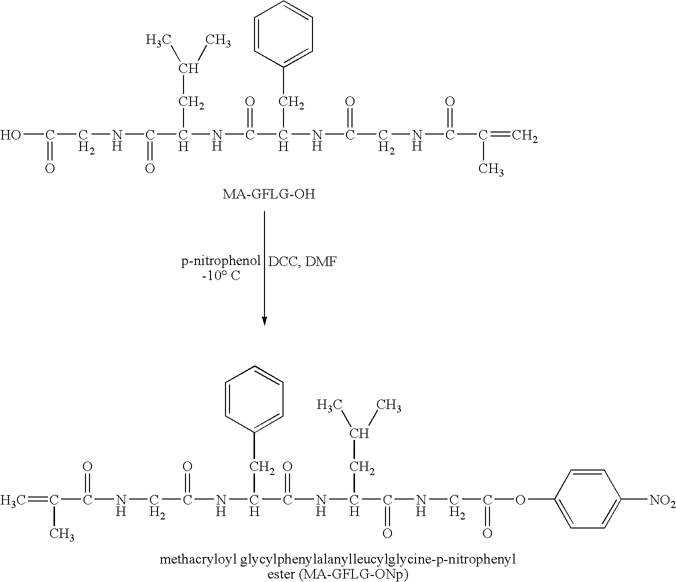

The extinction coefficient in DMSO (containing 1% acetic acid to prevent hydrolysis of the ONp-group) for the pure product is 9500 at λₘₐₓ=271 nm.

Copolymerization of MA-GFLG-ONp and HPMA

The polymerization was carried out using mixtures of HPMA, and MA-GFLG-ONp at various molar ratios using the initiator (2,2′-azobisisobutyronitrile, AIBN). The solution containing the monomers in desired molar ratios dissolved in acetone and mixed with the initiator was transferred to an ampoule and bubbled with nitrogen for 5 min. The ampoule was sealed and put in an oil bath at 50° C. for 24 hours under stirring. After 24 hours the copolymers would precipitate out of solution and the ampoules was cooled to room temperature and placed in the freezer for 20 min to increase the yield of the precipitated polymer further. The copolymers were filtered off, dissolved in methanol and reprecipitated in ether. After filtration and washing with ether the copolymers were dried under vacuum. The synthesized polymeric precursors were characterized by TLC and size exclusion chromatography. The content of the reactive p-nitrophenyl ester (ONp) was measured by WV spectrophotometry. The samples were dissolved in DMSO containing 1% acetic acid and the absorbance was measured at 271 nm.

Synthesis of HPMA-GFLG-DFMO Conjugate
Synthesis of copper-complex of DL-α-difluoromethylornithine (Cu-DFMO)

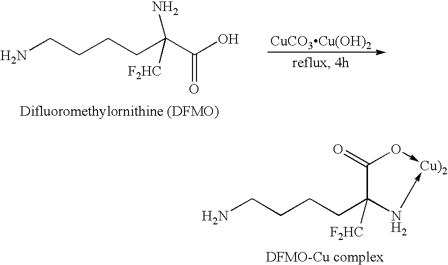

DL-α-difluoromethylornithine hydrochloride (DFMO.HCl) (90 mg, 0.41 mmol) was dissolved in 1 ml H₂O, and basic cupric carbonate (126 mg, 0.56 mmol) was added. The mixture was stirred and refluxed for 4 h. The resulting suspension was filtered and washed with hot H₂O until the filtrate was colorless. The combined filtrates were evaporated to dryness to give copper complexed DFMO. ESMS gave [M+H]⁺=425.7 Da, theoretical 425 Da
Synthesis of Polymer Conjugates

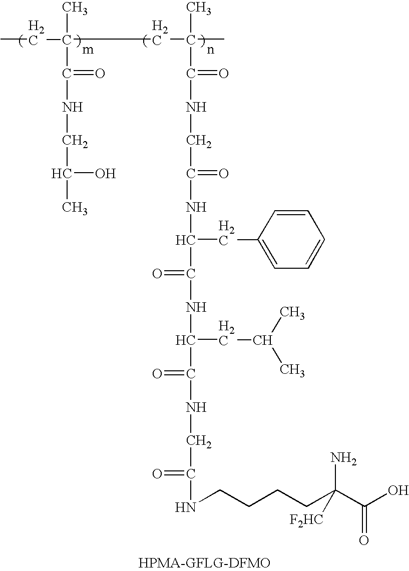

To a solution of HPMA-GFLG-ONp precursor (150 mg, containing 4 mole % ONp groups, 0.03 mmol) in 3 ml phosphate buffer (pH 7.2) was added a solution of Cu-DFMO (0.14 mmol NH₂ groups). The reaction mixture was stirred at room temperature for 16 h. Unreacted ONp groups were hydrolyzed by adding 2 μl aminopropanol. The reaction mixture (containing HPMA-GFLG-Cu-DFMO) was mixed with 1 ml of CH₃OH and stirred for 6 h. Na⁺ Chelex 100 resin (560 mg) was washed 6 times with 340 μl of 1 N acetic acid and 6 times with 340 μl of H₂O, then added to the reaction mixture-CH₃OH solution. The resin was stirred for 3 h at 20° C., filtered and washed 5 times each with 400 μl of CH₃OH—H₂O (1:1) and 500 μl H₂O. The combined filtrates were dialyzed for 2 days and lyophilized to get HPMA-GFLG-DFMO.

Determination of Molecular Weight and Molecular Weight Distribution of HPMA-GFLG Precursors

6 mg of HPMA-GFLG-ONp precursor was dissolved in 0.3 ml PBS (pH 7.4) and 0.3 ml 0.1N NaOH solution was added. The mixture was stirred for 10 min. 0.5 ml was applied on a Sephadex G-25 PD10 column (Amersham Biosciences) and eluted with PBS. The polymer fraction was collected in 2 ml and hydrolyzed ONp was fractionated. The molecular weight and molecular weight distribution of the polymer sample was estimated by size-exclusion chromatography, on a Superose 12 HR 10/30 column (Amersham) using a Fast Protein Liquid Chromatography (FPLC) instrument. The number average molecular weight (Mn), weight average molecular weight (Mw) and polydispersity (n) of the polymers were estimated from a calibration curve using polyHPMA fractions of known molecular weights. The molecular weights of a series of HPMA precursors containing 0, 2, 4, 6, 8 and 10 mol % ONp were estimated and is reported in Table 1

TABLE 1
Physicochemical characteristics of HPMA copolymer precursors.

| | ONp content | | | Polydis- |
|---|---|---|---|---|
| | (mmol/g | M_{w} | Mₙ | persity |
| Sample | polymer) | (g/mol) | (g/mol) | (n) |
| pHPMA | - (N/A) | 154000 | 148000 | 1.0 |
| HPMA-GFLG-ONp (2%) | 0.09 | 45700 | 31700 | 1.4 |
| HPMA-GFLG-ONp (4%) | 0.18 | 38000 | 28700 | 1.3 |
| HPMA-GFLG-ONp (6%) | 0.25 | 36900 | 25000 | 1.5 |
| HPMA-GFLG-ONp (8%) | 0.32 | 37600 | 25000 | 1.5 |
| HPMA-GFLG-ONp (10%) | 0.37 | 42800 | 25600 | 1.7 |

Determination of DFMO Content of RIPMA-GFLG-DFMO Conjugates

The DFMO content was estimated by estimating the amino groups in DFMO using trinitrobenzene sulfonate (TNBS). The following solutions were prepared:
Solution A: 100 ml of 0.1 M Na₂SO₃ (fresh each week)
Solution B: 1.01 of 0.1M NaH₂PO₄
Solution C: 1.01 of 0.1M Na₂B₄O₇ in 0.1M NaOH
Solution D: 1.5 ml of Solution A+98.5 ml of Solution B (fresh daily)
TNBS: 5% w/v solution

Sample solution was prepared in 0.25 ml H₂O. To it was added 0.25 ml solution C and 10 μl TNBS solution. The mixture was incubated for 5 min at 23° C. 1 ml of solution D was added to stop the reaction and the OD₄₂₀ was measured. The DFMO content of HPMA-GFLG-DFMO conjugate was estimated from the calibration curve of DFMO standards and expressed in mmol/g polymer conjugate (Table 2)

TABLE 2
Drug content of HPMA-GFLG-DFMO conjugate

| | ONp content | DFMO content | % ONp |
|---|---|---|---|
| Sample | (mmol/g polymer | (mmol/g polymer) | conversion |
| HPMA-GFLG- | 0.18 | 0.11 | 61 |
| DFMO (4%) | | | |

Synthesis of Methacryloyl-glycylphenylalanylleucylglycine-DFMO (MA-GFLG-DFMO)

To a solution of DFMO.HCl (37 mg, 0.17 mmol) in 0.3 ml DMSO was added dropwise a solution of MA-GFLG-ONp (67 mg, 0.11 mmol) in 0.5 ml DMSO, followed by triethylamine (24 μl, 0.17 mmol). The reaction was carried out at room temperature for 24 h and monitored by TLC for hydrolysis of ONp. The reaction mixture was rotavaped to remove DMSO and redissolved in methanol and precipitated in ether twice. The product was washed with ether and dried under vacuum. TLC (acetone:ether 3:7 v/v) showed no trace of free ONp. ESMS gave [M+H]⁺=625 Da, theoretical 624.3 Da.

Preparation of Complexes of Nucleic Acid with HPMA Conjugated Polyamine:

Preparation of complexes of nucleic acid will be performed using HPMA-conjugated polyamine and a variety of different nucleic acid forms. HPMA-conjugated polyamine will be used to compact plasmid DNA into a colloidal dispersion in water. The size and zeta potential of the colloidal dispersions prepared will be determined at different charge ratios for added cation (amine) to anion (DNA phosphate). The colloidal dispersions to be prepared enable binding the DNA into complexes that are suitable for the invention. Two critical factors will be examined, formulation with or without cholesterol and the ratio of cationic lipid to DNA. Additionally, the amount of Cholesterol will be tested over the range of 0.5:1 to 2:0.2 mole ratio of polyamine:chol with particular emphasis on 1:1 mole ratio of polyamine:chol.

Mixing

HPMA-conjugated polyamine will be dissolved in an aqueous solution to obtain a final concentration of 100 mM amine as determined by an ethidium bromide displacement assay. In this assay 1 mmol is defined as the amount of amine required to completely neutralize 1 mmol of DNA phosphate. From a 2.72 mg/ml stock solution of plasmid DNA (pCIluc) 221 μl will be combined with 110 μl of a concentrated aqueous solution of salts, buffers, detergents, etc. and 597 μl of water. 72 μl of the polyamine solution will be added to the mixture and vortexed thoroughly for 20 sec, to prepare complexes that have a 4:1+/− ratio. The particle size and distribution of size for each preparation made will be determined.

For a continuous process, streams of aqueous DNA, at a concentration of 50 μg/ml and of HPMA-conjugated polyamine will be fed into an HPLC static mixer which includes three 50 μl cartridges in tandem and the complexes collected from the output of the final mixer. In the making of each preparation of particles, each stream will be fed into the mixer at the same flow rate, and flow rate maintained as the resulting combined stream of DNA and polymer flows through the cartridges. Flow rates will be varied from 250 μl/min. to 5,000 μl/min. The procedure will be repeated, except that the streams of DNA and HPMA-polyamine will be fed into an HPLC mixer containing three 150 μl cartridges in tandem and flow rates varied from 500 μl/min. to 7,000 μl/min. The procedure will be repeated, except that the streams of DNA and HPMA-polyamine will contain one or more surfactant at one or more concentrations. In one case, Tween 80 detergent in an amount of 0.25% by volume will be added to the DNA stream prior to mixing with the polyamine. The procedure will be repeated, except that a mixture of HPMA-conjugated polyamine and polyamine that is not conjugated will be used to form the stream of polyamine. The procedure will be repeated, except that salt composition and concentrations added to the DNA and polyamine will be varied. In some instances, the preparation will be filtered through a 0.2μ filter and/or concentrated by ultrafiltration through an Amicon polysulfone (molecular weight 500 KDa) membrane at a flow rate of 300 μl/min. with isometric structure (Millipore Corporation, Bedford, Mass.) so that after concentration and filtration, the preparation will have an increased DNA concentration. The particle size and distribution of size for each preparation made will be determined.

The results will show that particle size can be adjusted by controlling one or more of the parameters including changing the size of the mixing cartridges, the flow rate, the concentration and ratio of the components, and the components of the aqueous phase. The results will show that the method is reproducible in that, when one mixes aqueous solutions of DNA and polyamine continuously at a constant charge ratio of polyamine to DNA at constant flow rates, one obtains homogenous preparations of particles of DNA and a polyamine consistently, wherein each preparation includes particles having similar mean particle sizes. Thus, one can choose conditions to provide particles of a desired concentration, size, and homogeneity. In addition, the ability to make such a preparation of complexes is independent of batch size.

Biological Activity Assay: Transfection

Transfection efficiency of polyamine conjugated HPMA/DNA complexes will be studied using a plasmid DNA, pCI-Luc containing Luciferase reporter gene, using a CMV promoter. Cells (B16) will be plated at 20000 cells/well in 96 well plates and allowed to grow to 80-90% confluency. They will then be incubated with polymer conjugate/DNA complexes prepared at a N/P ratio of 4 and a DNA dose of 0.5 (μg DNA per well, for 3 hours in serum free medium at 37° C. Cells will be allowed to grow in growth medium for another 20 hours before assaying for the Luciferase activity. Luciferase activity in terms of relative light units will be assayed using the commercially available kit (Promega) and read on a luminometer (Tropix, Applied Biosystems), using 96 well format

### Example A3

### Preparation of Lipoplexes containing DNA and HPMA-Conjugated Polyamine:

Forty microgram of pCILuc will be dissolved in 100 μl of aqueous phase, in one instance 10% glucose, and mixed by hand with an aqueous solution prepared with a mixture of HPMA-conjugated polyamine and an aqueous dispersion of cationic lipids in a final volume of 100 μl aqueous phase, in one instance distilled H2O. In this instance, the final concentration of glucose will be 5%. The concentration and ratio of the HPMA-conjugated polyamine and the cationic lipids will be varied. The mixing will be performed by adding the DNA solution to the lipid solution. The charge ratio of amine to DNA in this mixture will be varied by variation in the amounts of the lipids and HPMA-conjugated polyamine.

### Example A4

### Preparation of Ligand-Targeted Complexes Containing DNA and HPMA-Conjugated Polyamine:

Peptide K14RGD containing the amino acid sequence: KKK KKK KKK KKK KKS CRG DC, peptide K14SMT containing the amino acid sequence: KKK KKK KKK KKK KKA d-FCY d-WKT CT, and peptide K14MST containing the amino acid sequence KKK KKK KKK KKK KKA TDC RGE CF with at least 90% purity will be synthesized by solid phase synthesis. Peptides will be oxidized to make circularized peptide.

Complexes will be prepared as above except that varying amounts of ligand peptides will be mixed with polyamine. The mixture will be added to serum free medium containing pCIluc2 DNA. The complex will be incubated for 30 min before adding to the cells. 20000 HUVEC cells will be seeded to each well of a 96 well plate and cultured for 12 hr before transfection. The transfection solution will be removed after 3 hr and serum-containing medium added to the cells.

The results will show increased expression by addition of a peptide ligand to polyamine containing complexes.

Effect of HPMA-Conjugation on the Size and Stability of Polyamine/DNA Complexes

Complexes of HPMA-conjugated polyamine/DNA containing various molar concentration of HPMA will be prepared by hand mixing of equal volumes of DNA and aqueous HPMA-conjugated :polyamine with or without various additional basic (amine) materials, followed by vortexing for 30 to 60 seconds.

### Example A5

### Preparation of Ligand-Targeted-HPMA-Conjugated Polyamine Complexes

Ligand and polyamine conjugated methacryl polymer will be prepared by the co-polymerization of HPMA, MA-GG-polyamine and MA-peptide ligand (eg. Peptide containing RGD sequence flanked by other amino acids) mixed at various stochiometric ratios using an initiator of polymerization.

Synthesis of MA-ACRGDMFGCA

104.2 mg (0.1 mmol) of the peptide, ACRGDMFGCA will be dissolved in 1 ml of 0.1N NaOH (0.1 mmol) and cooled to 0° C. Freshly distilled MACl (15.6 mg, 0.15 mmol) in 1 ml of dichloromethane will be added dropwise up to room temperature and allowed to react for one hour. The pH will be maintained at around 6-7. The dichloromethane layer will be separated from the water layer, washed with 2 ml of water and discarded. The aqueous layer together with the washings will be mixed with 40 ml of EtOAc. Under vigorous stirring and cooling, HCl will be added slowly until the pH reached at 2-3. The organic layer will be separated and the aqueous layer will be extracted three times with EtOAc (3×20 ml). The extracted layers will be dried over anhydrous sodium sulfate overnight. The dried solution will be filtered and washed with EtOAc. The EtOAc will be removed by roto-evaporating to obtain the product as a white powder. Re-crystallization will be done from EtOAc.

Copolymerization of MA-GFLG-ONp, MA-ACRGDMFGCA and HPMA

The polymerization will be carried out using mixtures of HPMA, MA-GFLG-ONp and MA-ACRGDMFGCA at various molar ratios using the initiator (2,2′-azobisisobutyronitrile, AIBN). The solution containing the monomers in desired molar ratios dissolved in acetone and mixed with the initiator will be transferred to an ampoule and bubbled with nitrogen for 5 min. The ampoule will be sealed and put in an oil bath at 50° C. for 24 hours under stirring. After 24 hours the copolymers would precipitate out of solution and the ampoules will be cooled to room temperature and placed in the freezer for 20 min to increase the yield of the precipitated polymer further. The copolymers will be filtered off, dissolved in methanol and reprecipitated in ether. After filtration and washing with ether the copolymers will be dried under vacuum. The synthesized polymeric precursors will be characterized by TLC and size exclusion chromatography.

The content of the reactive p-nitrophenyl ester (ONp) will be measured by UV spectrophotometry. The samples will be dissolved in DMSO containing 1% acetic acid and the absorbance will be measured at 271 nm.

Synthesis of Poly(HPMA)-(ACRGDMFGCA)(GFLG-DFMO) Conjugate

(A 40 times molar excess of DFMO hydrochloride to ONp will be used to attach the polyamine to the polymeric backbone. The excess amount was used to minimize the well-established cyclization and crosslinking side reactions when diamines are reacted with reactive ester groups of water-soluble polymers (Ghandehari et al., 1996). To a 40 molar excess solution of DFMO hydrochloride in 15 ml of DMSO, the copolymers (0.6 g) in anhydrous DMSO (30 ml) will be added to undergo condensation. The reaction mixtures will be stirred at room temperature for 24 hours. After 24 hours the DMSO will be evaporated off and the residue will be dissolved in a small amount of methanol. The copolymer-drug conjugate will be precipitated in ether, filtered off, washed with ether and finally dried in vacuo.

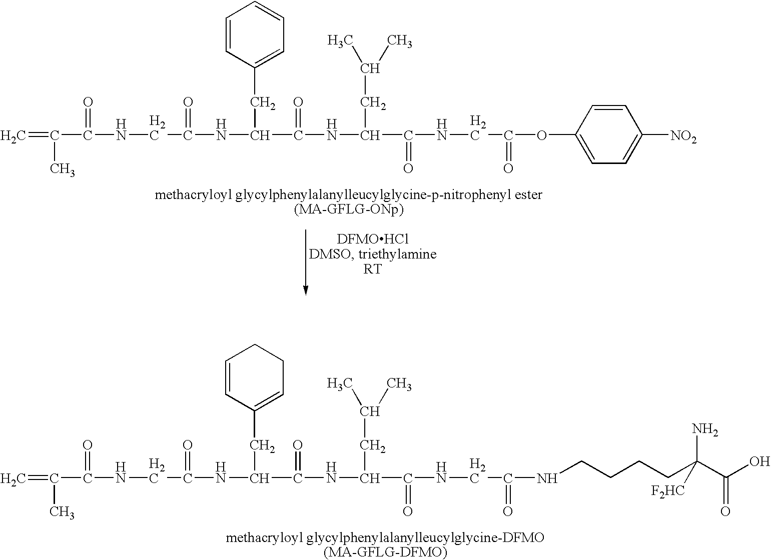

1. A composition for nucleic acid delivery, comprising HPMA conjugated to a polyamine.

2. The composition of claim 1, wherein said polyamine is selected from the group consisting of: spermine, spermidine and their analogues, and DFMO.

3. The composition of claim 1, further comprising a targeting ligand.

4. The composition of claim 1, further comprising one or more ligand binding domains.

5. The composition of claim 4, wherein the ligand binding domains are selected from the group consisting of: vascular endothelial growth factors, somatostatin and somatostatin analogs, transferring, melanotropin, ApoE and ApoE peptides, von Willebrand's factor and von Willebrand's factor peptides, adenoviral fiber protein and adenoviral fiber protein peptides, PD1 and PD1 peptides, EGF and EGF peptides, RGD peptides, CCK peptides, antibody and antibody fragments, folate, pyridoxyl and sialyl-LewisX and chemical analogs, DNA and RNA aptamers.

6. The composition of claim 5, wherein said antibody fragment is selected from the group consisting of: Fab′, F(ab′)₂, Fab, Facb, Fd, Fv, and scFv.

7. Then composition according to claim 1, further comprising a nucleic acid molecule.

8. A method for delivering a nucleic acid to an in vivo system, comprising administering to a subject an effective amount of a therapeutic nucleic acid bound to a polyamine that is conjugated to HPMA.

9. A method for inhibiting cellular proliferation, comprising administering an effective amount of a therapeutic nucleic acid via a polyamine conjugated to HPMA.

10. A method of vaccination comprising the steps of administering to a subject an effective amount of a therapeutic nucleic acid bound to a polyamine that is conjugated to HPMA and administering subsequent doses of the same therapeutic nucleic acid at intervals that optimize immune response.
